(19) United States
(12) Patent Application Publication
(10) Pub. No.: US 2004/0141919 A1
(43) Pub. Date: Jul. 22, 2004

(54) Method for detecting halitosis

(76) Inventor: John G. Robertson, Sarnich (CA)

Correspondence Address: LAHIVE & COCKFIELD, LLP., 28 STATE STREET, BOSTON, MA, 02109, US

(21) Appl. No.: 10/696,414

(22) Filed: Oct. 28, 2003

Related U.S. Application Data
(60) Provisional application No. 60/421,937, filed on Oct. 28, 2002.

(51) Int. Cl. A61K 49/00

(52) U.S. Cl. 424/9.1

(57) ABSTRACT

Methods and kits for the detection of halitosis are described.

### RELATED APPLICATIONS

This application claims priority to U.S. Provisional Patent Application Serial No. 60/421,937, filed on Oct. 28, 2002. The entire contents of the aforementioned provisional application are hereby incorporated herein by reference.

### BACKGROUND OF THE INVENTION

It is estimated that halitosis affects 60-65% of the population at one time or another (Spouge, J D, (1964), Dent. Practit. 14:307-317). Individuals suffering from halitosis experience considerable social and psychological discomfort. This results from the fact that the sufferer in general cannot determine when he or she is suffering from an acute episode and will rarely be told by others of his or her condition. This makes it virtually impossible for the user to determine the causes of his or her condition, treat the condition appropriately and to monitor the effectiveness of a particular treatment regimen.

In general, halitosis is caused by the production of various malodorous compounds from the metabolism of amino acids by gram-negative bacteria in the mouth (Kleinberg, I., Codipilly, M., (1997), Bad Breath Research Perspectives 2nd Ed, Ramot Pub, 13-39). While certain pathological conditions such as periodontal disease may cause or contribute to halitosis, many halitosis sufferers are healthy individuals with no known pathology (Yaegaki K., Sanada K, (1992) J. Peridont Res 27:233-238). It appears that non-pathogenic halitosis is caused by the actions of anaerobic bacteria residing on the rear portion of the tongue.

### SUMMARY OF THE INVENTION

The invention provides a simple, economical means by which subjects can monitor their breath for the malodorous constituents that produce halitosis. This may enable them to possibly discover the cause(s) of their halitosis, treat their condition appropriately when required, monitor treatment effectiveness and provide them with the confidence that will come from being able to manage their condition.

The invention provides, at least in part, to a method for detecting halitosis in a subject. The method includes providing an effective amount of at least one halitosis associated compound in a reservoir; and allowing the subject to smell the halitosis associated compound to detect an apparent odor, whereby the lack of an apparent odor of the halitosis associated compound to the subject is indicative of halitosis in the subject.

The invention also provides, at least in part, to a portable kit for the detection of halitosis. The kit comprises an effective amount of one or more halitosis associated compounds on a scratch and sniff substrate.

The invention also pertains, at least in part, to a portable kit for the detection of halitosis. The kit comprises an effective amount of two or more halitosis associated compounds in at least one reservoir.

### DETAILED DESCRIPTION OF THE INVENTION

Detection of halitosis has relied upon the measurement of volatile sulfur compounds (VSC) such as hydrogen sulfide, methyl mercaptan and dimethyl sulfide in the breath and mouth of the subject as these compounds appear to be associated with halitosis although other compounds such as indole and cadaversine may also contribute to breath malodour (Goldberg S, et al. (1997) Bad Breath Research Perspectives 2nd Ed, Ramot Pub, 71-85). However, there is evidence that methyl mercaptan is responsible for 90% of the malodour. Persons suffering from halitosis cannot detect their condition, possibly as their olfactory systems become desensitized to the malodorous substances in their breath.

The invention provides, at least in part, to a method for detecting halitosis in a subject. The method includes providing an effective amount of at least one halitosis associated compound in a reservoir; and allowing the subject to smell the halitosis associated compound to detect an apparent odor, whereby the lack of an apparent odor of the halitosis associated compound to the subject is indicative of halitosis in the subject.

The term “subject” includes organisms capable of suffering from halitosis and using the methods and kits of the invention. Preferably, the subject is a human.

The term “halitosis associated compound” includes substances known to produce the odors typical of halitosis. It also includes compounds which may smell similarly to compounds which contribute to halitosis. Compounds which smell similarly may be used if, for example, they are more easily handled, less reactive, or more stable. Examples of halitosis associated compounds include volatile sulfur compounds (VSC) such as hydrogen sulfide, methyl mercaptan, and dimethyl sulfide. Other examples also include indole and cadaversine.

The odor of the particular case of halitosis the subject is suffering from may vary depending upon the root cause of the halitosis, e.g. periodontal disease vs. non-pathogenic origins. Therefore, the invention also may further comprise two or more reservoirs containing different halitosis associated compounds and/or different mixtures of halitosis associated compounds.

The term “effective amount” includes amounts of halitosis associated compounds such that a subject not suffering from halitosis will be able to detect an odor whilst a subject suffering from halitosis will detect no odor. Preferably, the person not suffering from halitosis will only detect a slight odor of the halitosis associated compound.

The term “reservoir” includes materials which are capable of containing an effective amount of at least one halitosis associated compound. In one embodiment, the reservoir is a scratch and sniff substrate. In another embodiment, the reservoir comprises glass, plastic, ceramic, paper, or metal. In one embodiment, the reservoir is a bottle, vial, or a plate with one or more wells.

The term “scratch and sniff substrate” includes the substrates and scent delivery materials described in U.S. Pat. No. 6,248,377; U.S. Pat. No. 5,640,931, U.S. Pat. No. 4,983,404, U.S. Pat. No. 5,039,243, and U.S. Pat. No. 5,018,974. Other scent delivery methods are also known to those of skill in the art.

The invention also provides, at least in part, to a kit for the detection of halitosis. The kit comprises an effective amount of one or more halitosis associated compounds on a scratch and sniff substrate.

The invention also pertains, at least in part, to a portable kit for the detection of halitosis. The kit comprises an effective amount of two or more halitosis associated compounds in at least one reservoir.

The term “portable” includes kits which can be carried and do not require an external power supply. In one embodiment, the portable kit weighs under 5 pounds, under 4 pounds, under 3 pounds, under 2 pounds, under 1 pound, or under 8 ounces. Previous devices for halitosis detection have been developed, but these devices are expensive and require some training (Yaegaki, K, (1997) Bad Breath Research Perspectives 2nd Ed, Ramot Pub, 227-231; Diamond et al. U.S. Pat. No. 6,264,615). While portable in the sense that they can be transported from one location to another they require an external power source and are not small enough, for example, to be carried in a pocket or purse.

The kits of the invention, as described above, provide a reservoir or reservoirs comprising effective amounts of one or more of the halitosis associated compounds. The effective amounts of the halitosis associated compounds may be selected such that a person not suffering from halitosis will be able to detect only slight odor whilst the person suffering from halitosis will detect no odor. The reservoirs may comprise a variety of containers such as stoppered glass or plastic bottles or scratch and sniff substrates.

To use the portable kit, a subject sniffs the exposed the contents of the reservoir(s). If odors are detected from the reservoirs, then the subject would know that he or she is free of halitosis. Conversely, if the subject is unable to detect one or more of the odors, it would indicate that the person is suffering from an acute episode of halitosis.

Equivalents

Those skilled in the art will recognize, or be able to ascertain using no more than routine experimentation, many equivalents to the specific embodiments and methods described herein. Such equivalents are intended to be encompassed by the scope of the following claims. The entire contents of all references and patents cited herein are hereby incorporated by reference.

1. A method for detecting halitosis in a subject, comprising:
providing an effective amount of at least one halitosis associated compound in a reservoir;
allowing said subject to smell said halitosis associated compound to detect an apparent odor, whereby the lack of an apparent odor of said halitosis associated compound to said subject is indicative of halitosis in said subject.

2. The method of claim 1, wherein said halitosis associated compound is a volatile sulfur compound.

3. The method of claim 2, wherein said volatile sulfur compound is hydrogen sulfide, methyl mercaptan, or dimethyl sulfide.

4. The method of claim 1, wherein said halitosis associated compound is indole or cadaversine.

5. The method of claim 1, wherein said reservoir is a scratch and sniff substrate.

6. The method of claim 1, further comprising providing an effective amount of two or more halitosis associated compounds.

7. The method of claim 1, wherein said reservoir is glass, plastic, paper, metal, or ceramic.

8. A kit for the detection of halitosis, comprising:
an effective amount of one or more halitosis associated compounds on a scratch and sniff substrate.

9. A portable kit for the detection of halitosis, comprising:
an effective amount of two or more halitosis associated compounds in at least one reservoir.

10. The portable kit of claim 8 or 9, wherein said halitosis associated compound is a volatile sulfur compound.

11. The portable kit of claim 10, wherein said volatile sulfur compound is hydrogen sulfide, methyl mercaptan, or dimethyl sulfide.

12. The portable kit of claim 8 or 9, wherein said halitosis associated compound is indole or cadaversine.

13. The portable kit of claim 9, wherein said reservoir is plastic, glass, paper, or ceramic.
